(19) United States
(12) Patent Application Publication
(10) Pub. No.: US 2024/0153642 A1
(43) Pub. Date: May 9, 2024

(54) PROCESSING DEVICE, NON-TRANSITORY COMPUTER READABLE STORAGE MEDIUM, METHOD FOR GENERATING TRAINING DATA, AND METHOD FOR GENERATING PREDICTION MODEL

(71) Applicant: NIHON KOHDEN CORPORATION, Tokyo (JP)

(72) Inventors: Yota SEKIGUCHI, Tokorozawa-shi (JP); Takuya UMEMOTO, Tokorozawa-shi (JP); Keiichi SATO, Tokorozawa-shi (JP); Wataru MATSUZAWA, Tokorozawa-shi (JP)

(73) Assignee: NIHON KOHDEN CORPORATION, Tokyo (JP)

(21) Appl. No.: 18/498,532

(22) Filed: Oct. 31, 2023

(30) Foreign Application Priority Data
Nov. 7, 2022 (JP) 2022-177944

(51) Int. Cl. G16H 50/30

(52) U.S. Cl. CPC G16H 50/30

(57) ABSTRACT

A processing device includes an interface configured to receive electrocardiographic waveform data corresponding to an electrocardiographic waveform of a subject, and a processor configured to divide the electrocardiographic waveform into a plurality of partial electrocardiographic waveforms, based on the electrocardiographic waveform data, calculate a probability that a waveform portion in which a heart disease is suspected is included in each of the plurality of partial electrocardiographic waveforms, and cause a display to display an index together with information, the index corresponding to the probability, the information corresponding to the partial electrocardiographic waveform from which the probability is calculated.

### CROSS-REFERENCE TO RELATED APPLICATIONS

This application is based on and claims priority under 35 USC 119 from Japanese Patent Application No. 2022-177944 filed on Nov. 7, 2022, the entire content of which is incorporated herein by reference.

### TECHNICAL FIELD

The presently disclosed subject matter relates to a processing device configured to perform a processing of calculating a probability that a waveform portion in which a heart disease is suspected is included in an electrocardiographic waveform acquired from a subject, and a non-transitory computer readable storage medium storing a computer program configured to be executed by a processor mounted on the processing device. The presently disclosed subject matter also relates to a method for generating training data for machine-learning a prediction model used for calculation of a probability that a waveform portion in which a heart disease is suspected is included in an electrocardiographic waveform acquired from a subject, and a method for generating the prediction model.

### BACKGROUND ART

JP2014-054391A discloses a Holter electrocardiograph that acquires an electrocardiographic waveform of a subject. Data corresponding to the electrocardiographic waveform is received by an information processing device that is remotely arranged. The information processing device is configured to analyze the data to determine whether a waveform portion in which a heart disease is suspected is included in the electrocardiographic waveform.

It is desired to provide more detailed information regarding whether a waveform portion in which heart disease is suspected is included in the electrocardiographic waveform acquired from the subject.

### SUMMARY OF INVENTION

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to a first aspect of the present disclosure, there is provided a processing device including:
an interface configured to receive electrocardiographic waveform data corresponding to an electrocardiographic waveform of a subject; and
a processor configured to:
   divide the electrocardiographic waveform into a plurality of partial electrocardiographic waveforms, based on the electrocardiographic waveform data;
   calculate a probability that a waveform portion in which a heart disease is suspected is included in each of the plurality of partial electrocardiographic waveforms; and
   cause a display to display an index together with information, the index corresponding to the probability, the information corresponding to the partial electrocardiographic waveform from which the probability is calculated.

According to a second aspect of the present disclosure, there is provided a non-transitory computer readable storage medium storing a computer program , the computer program including instructions which, when executed by a processor mounted on a processing device, cause the processing device to:
receive electrocardiographic waveform data corresponding to an electrocardiographic waveform of a subject;
divide the electrocardiographic waveform into a plurality of partial electrocardiographic waveforms, based on the electrocardiographic waveform data;
calculate a probability that a waveform portion in which a heart disease is suspected is included in each of the plurality of partial electrocardiographic waveforms; and
cause a display to display an index together with information, the index corresponding to the probability, the information corresponding to the partial electrocardiographic waveform from which the probability is calculated.

According to a third aspect of the present disclosure, there is provided a method for generating training data for machine-learning a prediction model, the prediction model being used for calculating a probability that a waveform portion in which a heart disease is suspected is included in an electrocardiographic waveform acquired from a subject, the method including:
acquiring electrocardiographic waveform data corresponding to the electrocardiographic waveform including the waveform portion in which the heart disease is suspected;
acquiring beat information on the subject from the electrocardiographic waveform; and
generating a data set in which the electrocardiographic waveform data is associated with data corresponding to the beat information.

According to a fourth aspect of the present disclosure, there is provided a method for generating a prediction model, the prediction model being used for calculating a probability that a waveform portion in which a heart disease is suspected is included in an electrocardiographic waveform acquired from a subject, the method including:
acquiring training data generated by the method according to claim 10; and
performing machine learning using the training data.

According to a fifth aspect of the present disclosure, there is provided a method for generating a prediction model, the prediction model being used for calculating a probability that a waveform portion in which a heart disease is suspected is included in an electrocardiographic waveform acquired from a subject, the method including:
acquiring a data set generated by the processing device according to claim 7; and
performing machine learning using the data set.

### BRIEF DESCRIPTION OF DRAWINGS

Exemplary embodiment(s) of the present invention will be described in detail based on the following figures, wherein:

FIG. 1 illustrates a functional configuration of a processing device according to an exemplary embodiment of the present invention;

FIG. 2 illustrates a procedure of processing performed by a processor of FIG. 1;

FIG. 3 illustrates an index displayed on a display by the processing of FIG. 2;

FIG. 4 illustrates an example of a screen displayed on the display by the processing of FIG. 2;

FIG. 5 illustrates another example of the screen displayed on the display by the processing of FIG. 2;

FIG. 6 illustrates an example of a processing of changing a displayed index;

FIG. 7 illustrates other example of the processing of changing the displayed index;

FIG. 8 illustrates other example of the processing of changing the displayed index;

FIG. 9 illustrates a functional configuration of a device configured to generate a prediction model of FIG. 1;

FIG. 10 illustrates an example of a configuration of a processing system including the processing device of FIG. 1;

FIG. 11 illustrates another example of the configuration of the processing system including the processing device of FIG. 1; and

FIG. 12 illustrates a data set that can be used in training data of FIG. 9.

### DESCRIPTION OF EMBODIMENTS

An embodiment will be described in detail with reference to the accompanying drawings.

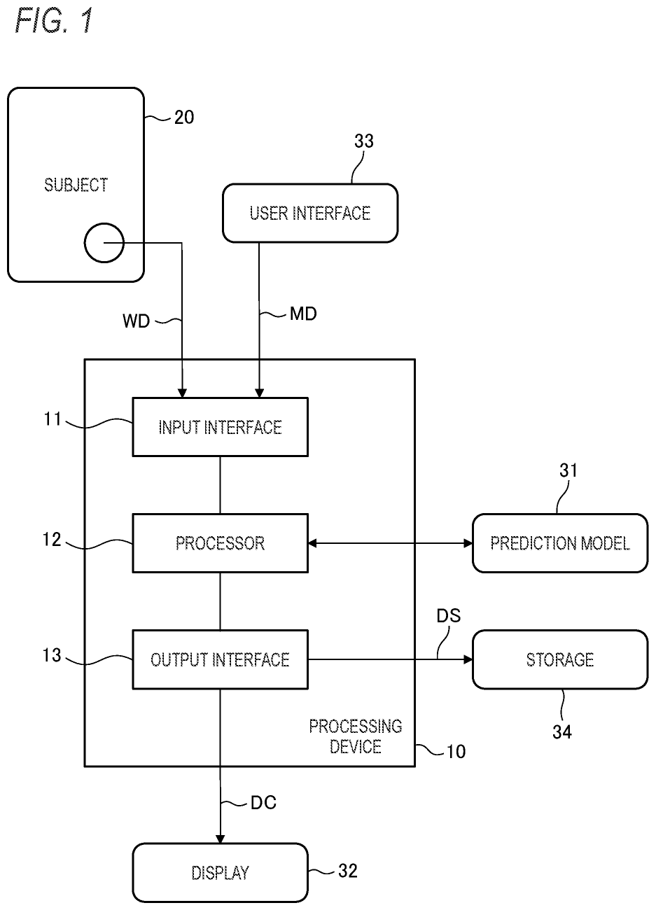

FIG. 1 illustrates a functional configuration of a processing device 10 according to an embodiment. The processing device 10 is configured to perform a processing of calculating a probability that a waveform portion in which atrial fibrillation is suspected is included in an electrocardiographic waveform acquired from a subject 20. An irregular pulse including the atrial fibrillation is an example of a heart disease.

The processing device 10 can include an input interface 11. The input interface 11 is configured as a hardware interface configured to receive electrocardiographic waveform data WD corresponding to the electrocardiographic waveform of the subject 20 acquired through an electrocardiograph or the like. The electrocardiographic waveform data WD may be digital data or analog data, in accordance with the specification of an electrocardiograph or the like.

In a case where the electrocardiographic waveform data WD is the analog data, the input interface 11 can include an appropriate conversion circuit including an A/D converter. This description is same or similarly applied to other signals and data that can be received by the input interface 11 to be described later.

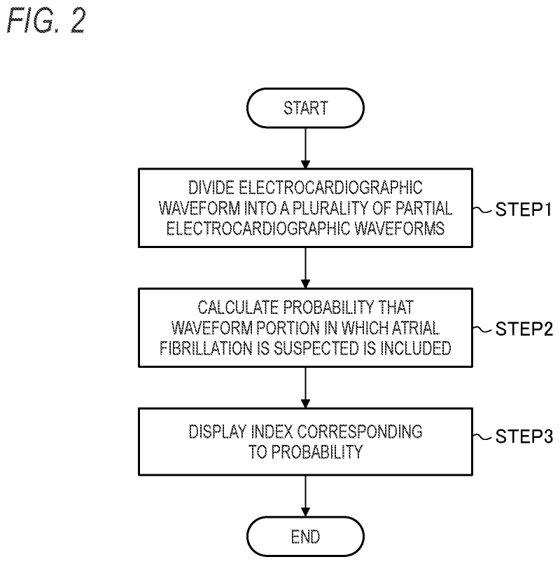

The processing device 10 can include one or more processors 12. The processor 12 is configured as an arithmetic element configured to perform the processing illustrated in FIG. 2.

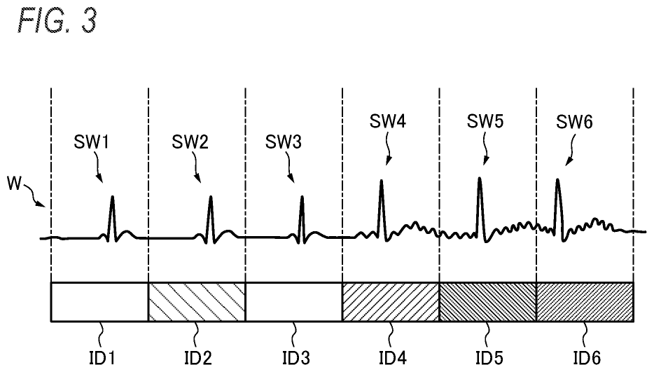

First, a processing corresponding to dividing the acquired electrocardiographic waveform into a plurality of partial electrocardiographic waveforms is performed on the electrocardiographic waveform data WD (STEP 1). Each of the partial electrocardiographic waveforms has a predetermined time length. The predetermined time length is, for example, 1 second. In an example illustrated in FIG. 3, an electrocardiographic waveform W includes six partial electrocardiographic waveforms SW1 to SW6.

Subsequently, a processing of calculating a probability that each of the plurality of partial electrocardiographic waveforms includes a waveform portion in which atrial fibrillation is suspected is performed (STEP 2 in FIG. 2). Specifically, the processor 12 is configured to input, to a prediction model 31 illustrated in FIG. 1, data corresponding to each of the partial electrocardiographic waveforms.

Note that the input of data to the prediction model 31 does not necessarily have to be performed in units for each of the divided partial electrocardiographic waveforms. Data corresponding to a plurality of partial electrocardiographic waveforms may be collectively input to the prediction model 31.

The prediction model 31 is a prediction algorithm generated through machine learning to be described later. The prediction model 31 is configured to output, as a predicted result, the probability that the waveform portion in which the atrial fibrillation is suspected is included in the partial electrocardiographic waveform, using the data corresponding to the partial electrocardiographic waveform as an input. The probability takes a value from 0 to 1. The value 0 corresponds to 0%. The value 1 corresponds to 100%. The predicted result may be a score (for example, any value from 1 to 5) corresponding to the predicted probability.

Subsequently, a processing of causing a display 32 illustrated in FIG. 1 to display an index corresponding to the above probability together with the partial electrocardiographic waveform in which the probability is calculated is performed (STEP 3 in FIG. 2).

As illustrated in FIG. 1, the processing device 10 can include an output interface 13.

The processor 12 is configured to output, from the output interface 13, a display control signal DC for causing the display 32 to display the electrocardiographic waveform W. The output interface 13 is configured as a hardware interface. The display control signal DC may be a digital signal or an analog signal, in accordance with the specification of the display 32.

In a case where the display control signal DC is the analog signal, the output interface 13 includes an appropriate conversion circuit including a D/A converter. This description is same or similarly applied to other signals and data that can be output by the output interface 13 to be described later.

As illustrated in FIG. 3, the display control signal DC is configured to cause the display 32 to display a plurality of indexes ID1 to ID6 side by side with the electrocardiographic waveform W. Each of the plurality of indexes ID1 to ID6 is displayed to be aligned with the corresponding one of the plurality of partial electrocardiographic waveforms SW1 to SW6.

In the present example, the index is a color of a strip-shaped graphic. In the present example, the color of the graphic is any one of four colors corresponding to the calculated probability, in which colorless is also an example of a color.

In the example illustrated in FIG. 3, a probability indicated by the index ID2 associated with the partial electrocardiographic waveform SW2 is higher than probabilities indicated by the indexes ID1 and ID3 associated with the partial electrocardiographic waveforms SW1 and SW3. The probability indicated by the index ID4 associated with the partial electrocardiographic waveform SW4 is higher than the probability indicated by the index ID2 associated with the partial electrocardiographic waveform SW2. Probabilities indicated by the indexes ID5 and ID6 associated with the partial electrocardiographic waveforms SW5 and SW6 are higher than the probability indicated by the index ID4 associated with the partial electrocardiographic waveform SW4.

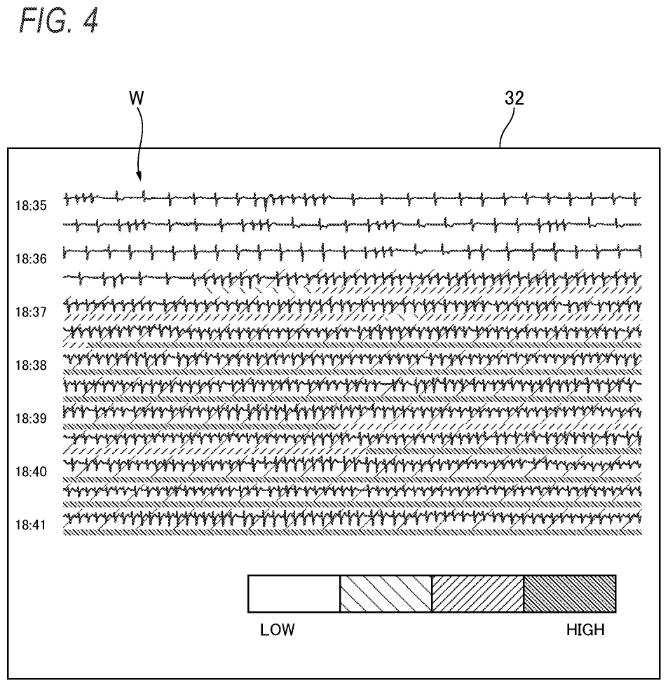

FIG. 4 illustrates an example of a screen displayed on the display 32 based on the display control signal DC. The electrocardiographic waveform W acquired for 6 minutes is displayed. Below each of the partial electrocardiographic waveforms included in the electrocardiographic waveform W, the above index is displayed so as to correspond thereto.

In other words, instead of extracting only the partial electrocardiographic waveform associated with the specific probability and displaying the extracted partial electrocardiographic waveform on the display device 32, the electrocardiographic waveform W including all the partial electrocardiographic waveforms is used for display. The index associated with each of all the partial electrocardiographic waveforms included in the electrocardiographic waveform W is displayed together with the electrocardiographic waveform W.

According to such a configuration, it is possible to provide a bird's-eye view point to the user about how portions that are predicted to have a high (or low) probability that a waveform portion in which the atrial fibrillation is suspected is included in a change over time of the electrocardiographic waveform W are distributed. In other words, it is possible to provide more detailed information regarding whether a waveform portion in which the atrial fibrillation is suspected is included in the electrocardiographic waveform W acquired from the subject 20.

It is possible to provide the user with a confirmation method in which the validity of prediction is confirmed more intensively for a portion that is predicted to have a medium probability that a waveform portion in which the atrial fibrillation is suspected in included. That is, it is possible to improve the efficiency of a work of confirming whether the waveform portion in which the atrial fibrillation is suspected is included in the electrocardiographic waveform W acquired from the subject 20.

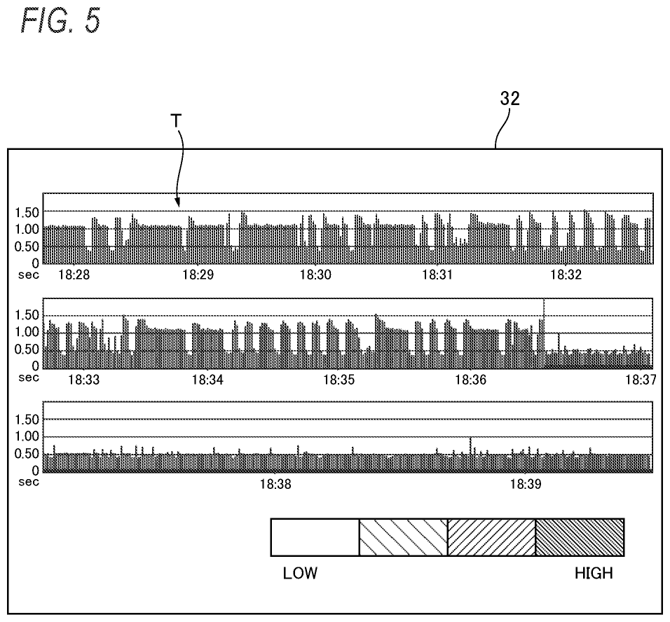

FIG. 5 illustrates another example of the screen displayed on the display 32, based on the display control signal DC. The display control signal DC according to the present example is configured to cause the display 32 to display a tachogram T indicating a change over time of an R-R interval in the electrocardiographic waveform W. The tachogram T is an example of information corresponding to the partial electrocardiographic waveform. The indexes described above are displayed to correspond to lower sides of the portions corresponding to the respective partial electrocardiographic waveforms included in the tachogram T.

As illustrated in FIG. 1, the input interface 11 of the processing device 10 may be configured to receive correction instruction data MD input through a user interface 33. In a case where the user such as a medical worker determines that the index displayed on the display 32 needs to be changed through the above prediction, the user can input an instruction corresponding to a desired change to the user interface 33. The correction instruction data MD includes data for changing an index displayed on the display 32, based on the instruction.

For example, in a case where the user desires to change the color of the index ID3 illustrated in FIG. 3 to the color of the index ID2, the correction instruction data MD can include data designating the index ID3 and data designating the color after the change.

The processor 12 is configured to output, from the output interface 13, the display control signal DC for causing the display 32 to change the index displayed based on the correction instruction data MD. In the above example, the display control signal DC for matching the color of the index ID3 with the color of the index ID2 is output from the output interface 13.

According to such a configuration, it is possible to provide an editability for a

predicted result by the processor 12, regarding whether a waveform portion in which the atrial fibrillation is suspected is included in the electrocardiographic waveform W acquired from the subject 20. It is possible to further improve convenience by allowing intervention of a user such as a medical worker with respect to the predicted result.

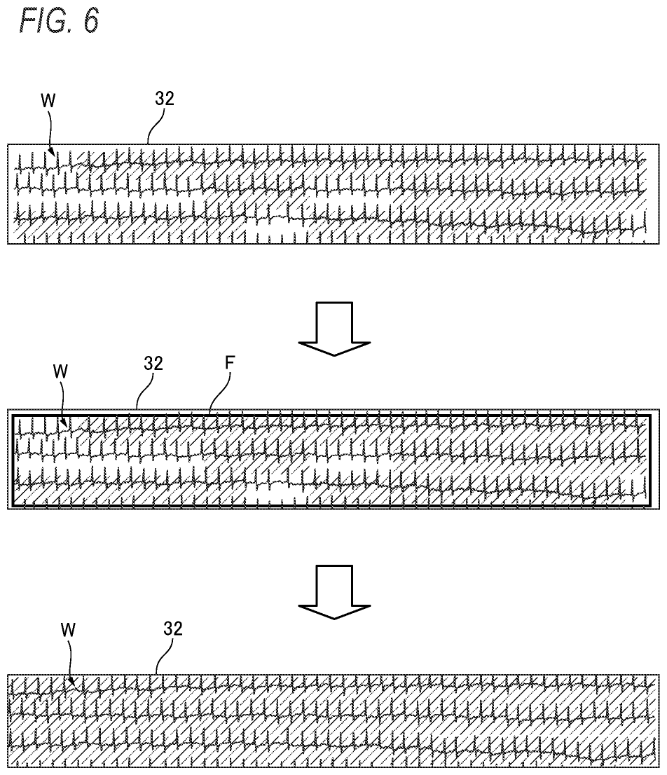

FIG. 6 illustrates another example of a change to the predicted result. In the present example, the index corresponding to the probability that the waveform portion in which the atrial fibrillation is suspected is included in the electrocardiographic waveform W acquired from the subject 20, is the color of a background of the partial electrocardiographic waveform. In the present example, the background of the partial electrocardiographic waveform in which the probability is predicted to be less than a predetermined threshold value is colorless, and the background of the partial electrocardiographic waveform in which the probability is predicted to be equal to or greater than the threshold value is colored.

On the screen of the display 32 at a certain time point, a colorless index and a colored index are mixed and displayed. A user such as a medical worker can change the color of the index to one of the colors. Specifically, a frame F for designating an area desired to be changed on the screen is set through the user interface 33. In other words, a specific time section in the electrocardiographic waveform W displayed on the display 32 is designated by the frame F. The correction instruction data MD corresponding to the setting is output from the user interface 33 and received by the input interface 11 of the processing device 10.

The processor 12 is configured to specify an index having a higher occupancy rate among the indexes included in the time section (the area surrounded by the frame F) designated by the correction instruction data MD, and is configured to output, from the output interface 13, the display control signal DC for causing the display 32 to change the index to match another index.

In the example illustrated in FIG. 6, the colored index has a higher occupancy rate. In accordance with this, the processor 12 outputs, from the output interface 13, the display control signal DC for causing the display 32 to change the colorless index to the colored index.

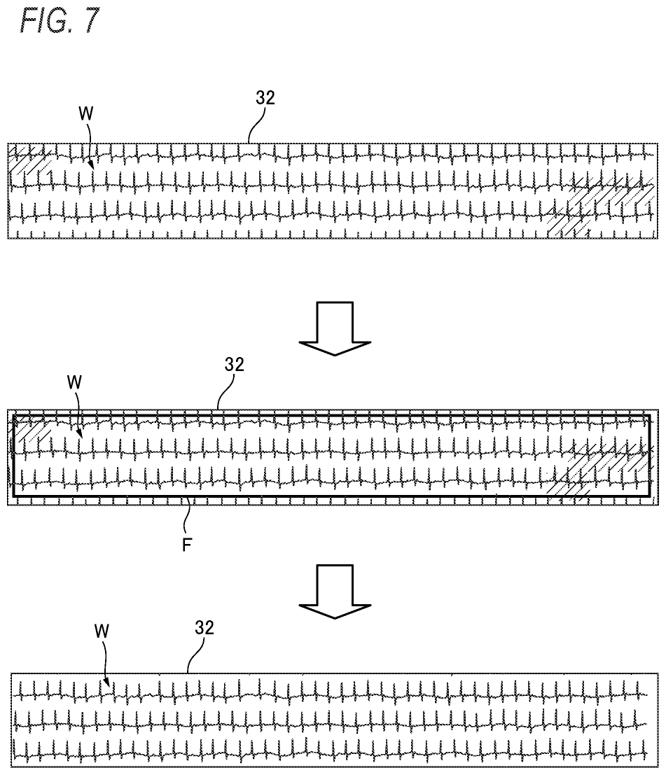

In the example illustrated in FIG. 7, the colorless index has a higher occupancy rate. In accordance with this, the processor 12 outputs, from the output interface 13, the display control signal DC for causing the display 32 to change the colored index to the colorless index.

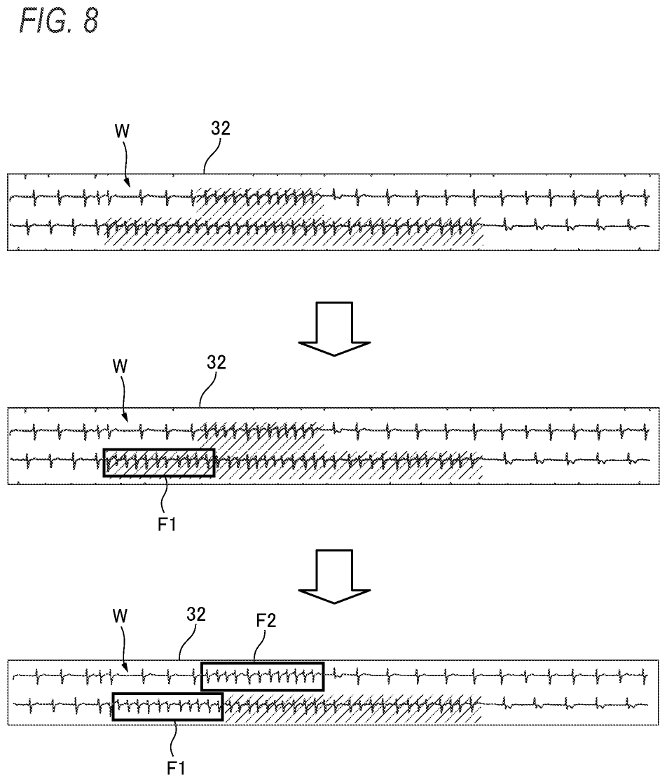

FIG. 8 illustrates other example of a change to the predicted result. In the present example, a user such as a medical worker sets a frame F1 for designating an area in which the color of the index is desired to be changed through the user interface 33. In other words, a specific time section in the electrocardiographic waveform W displayed on the display device 32 is designated by the frame F1. The correction instruction data MD corresponding to the setting is output from the user interface 33 and received by the input interface 11 of the processing device 10.

The processor 12 is configured to specify a waveform similar to a partial electrocardiographic waveform included in the designated time section, among the partial electrocardiographic waveforms not included in the time section designated by the correction instruction data MD (the area surrounded by the frame F1). In the example illustrated in FIG. 8, an area surrounded by a frame F2 is specified as a similar partial electrocardiographic waveform. Subsequently, the processor 12 outputs, from the output interface 13, the display control signal DC for causing the display 32 to change the color of the index associated with the partial electrocardiographic waveform specified as the similar partial electrocardiographic waveform together with the color of the index associated with the partial electrocardiographic waveform included in the designated time section. In the example illustrated in FIG. 8, each of the index surrounded by the frame F1 and the index surrounded by the frame F2 is changed from colored to colorless.

According to the configuration described with reference to FIGS. 6 to 8, it is possible to increase the efficiency of a correction work particularly in a case where there are a plurality of portions recognized as erroneous estimation.

In a case where the index is changed by the user as described above, the processor 12 may be configured to generate a data set DS in which the data corresponding to the changed index is associated with a part of the electrocardiographic waveform data WD corresponding to the partial electrocardiographic waveform specified by the changed index.

As illustrated in FIG. 1, the processor 12 can output the generated data set DS from the output interface 13, in order to store the data set DS in a storage 34. The storage 34 is a storage device that can be realized by a semi-conductor memory, a hard disk device, a magnetic tape device, or the like.

The fact that a certain index has been corrected means that there is room for correction in the predicted result with respect to the probability that the waveform portion in which the atrial fibrillation is suspected is included in the partial electrocardiographic waveform specified by the index. In other words, this means that there is room for improvement in the prediction algorithm of the prediction model 31. By collecting and accumulating the data set DS as described above, it is possible to use the data set DS as training data in a case where re-learning of the prediction model 31 is required. As a result, it is possible to improve the prediction accuracy of the probability that the waveform portion in which the atrial fibrillation is suspected is included in the electrocardiographic waveform W acquired from the subject 20 through improvement of the prediction model 31. Note that the data set DS as described above may be used as the training data at the time of initial learning of the prediction model 31.

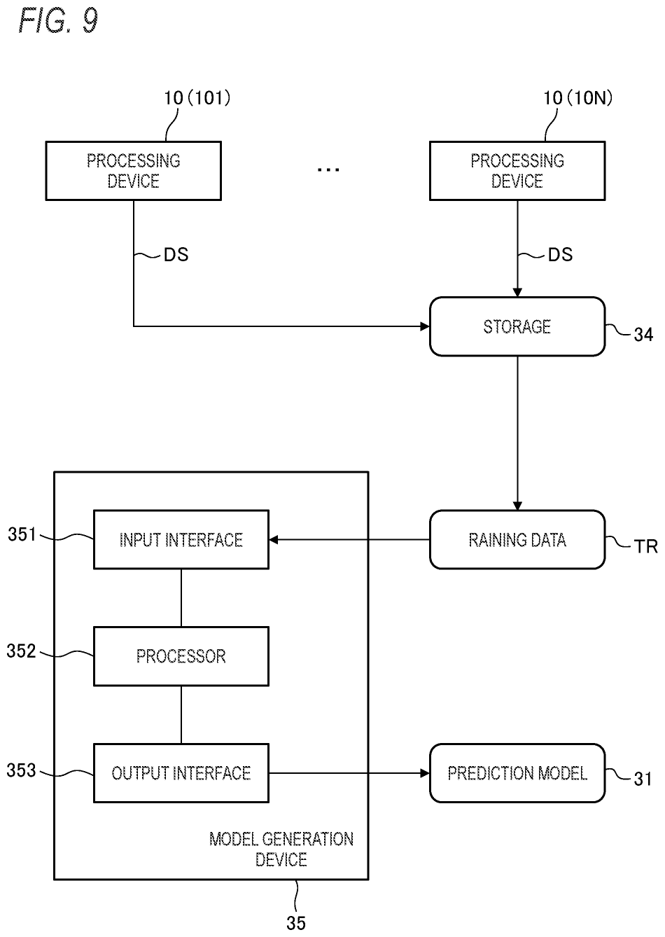

The prediction model 31 can be generated by a model generation device 35 illustrated in FIG. 9. That is, the model generation device 35 is configured to generate the prediction model 31 implemented in the above processing device 10.

The model generation device 35 can include an input interface 351. The input interface 351 is configured as a hardware interface configured to receive the training data TR. The training data TR can be acquired from the storage 34.

As an example, the training data TR is a data set in which data corresponding to an electrocardiographic waveform acquired from a certain subject is associated with training label data indicating whether the subject has caused atrial fibrillation.

As another example, the training data TR is a data set in which data corresponding to the beat information acquired from the above electrocardiographic waveform is associated with the training label data indicating whether the subject has caused the atrial fibrillation. Examples of the beat information include R-R intervals, normal beats, and supraventricular extrasystole. Since the data corresponding to the beat information is smaller in data size than the data corresponding to the electrocardiographic waveform, the calculation load related to the machine learning can be reduced. In addition, the diversity of the machine learning can be enhanced by preparing the data corresponding to the beat information that is difficult to directly acquire from the electrocardiographic waveform separately from the data corresponding to the electrocardiographic waveform.

In accordance with this, in order to generate the training data TR according to other example, the electrocardiographic waveform data corresponding to the electrocardiographic waveform of the subject is acquired, desired beat information is acquired from the electrocardiographic waveform, and the electrocardiographic waveform data and the data corresponding to the beat information are associated as a data set. The generated training data TR is stored in the storage 34.

The model generation device 35 can include one or more processors 352. The processor 352 is configured to generate the prediction model 31 by performing the machine learning using the training data TR. Examples of algorithms used for the machine learning can include a neural network, a decision tree, a random forest, and a support vector machine.

The model generation device 35 can include an output interface 353. The output interface 353 is configured as a hardware interface configured to output the prediction model 31 generated by the processor 352 in a form that can be implemented in the processing device 10.

The training data TR configured as described above is used to teach what kind of electrocardiographic waveform or beat information is acquired from the subject to determine that the subject is causing or not causing the atrial fibrillation. By generating the prediction model 31 that has been subjected to the machine learning using the training data TR in accordance with this, it is possible to increase the accuracy of the probability that the waveform portion in which the atrial fibrillation is suspected is included in the electrocardiographic waveform W acquired from the subject 20 that is predicted by the processing device 10.

The processing device 10, the prediction model 31, the display device 32, the user interface 33, and the storage 34 described above may be included in a processing system 40 configured to perform the processing of calculating the probability that the waveform portion in which the atrial fibrillation is suspected is included in the electrocardiographic waveform W acquired from the subject 20. The processing system 40 may have various configurations.

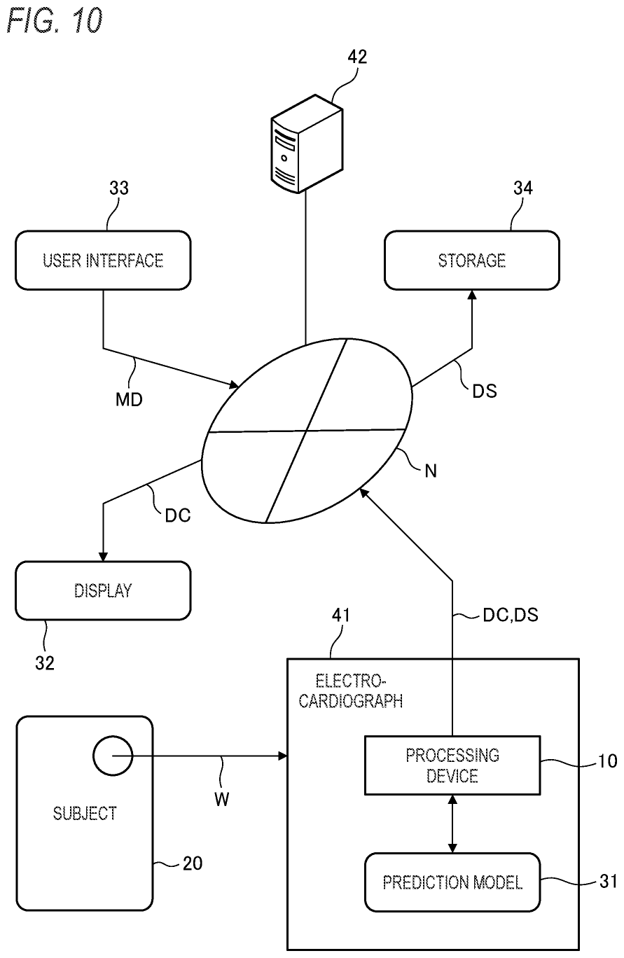

FIG. 10 illustrates an example of a configuration of the processing system 40. The processing system 40 can include an electrocardiograph 41. The electrocardiograph 41 is a device configured to acquire the electrocardiographic waveform W from the subject 20 and is configured to generate the corresponding electrocardiographic waveform data WD.

The processing device 10 and the prediction model 31 are mounted on the electrocardiograph 41. Each of the display device 32, the user interface 33 and the storage 34 is communicably connected to the processing device 10 via a communication network N.

The processing system 40 can include a server device 42. The server device 42 is communicably connected to the processing device 10 via the communication network N. The prediction model 31 may be mounted on the server device 42. In this case, the processor 12 of the processing device 10 is configured to transmit, from the output interface 13 to the server device 42, a part of the electrocardiographic waveform data WD corresponding to the partial electrocardiographic waveform, and configured to receive data corresponding to the probability calculated by the prediction model 31 through the input interface 11.

The display 32 may be mounted on the electrocardiograph 41. The user interface 33 may be mounted on the display 32 or may be mounted on the electrocardiograph 41. The storage 34 may be mounted on the electrocardiograph 41 or may be mounted on the server device 42.

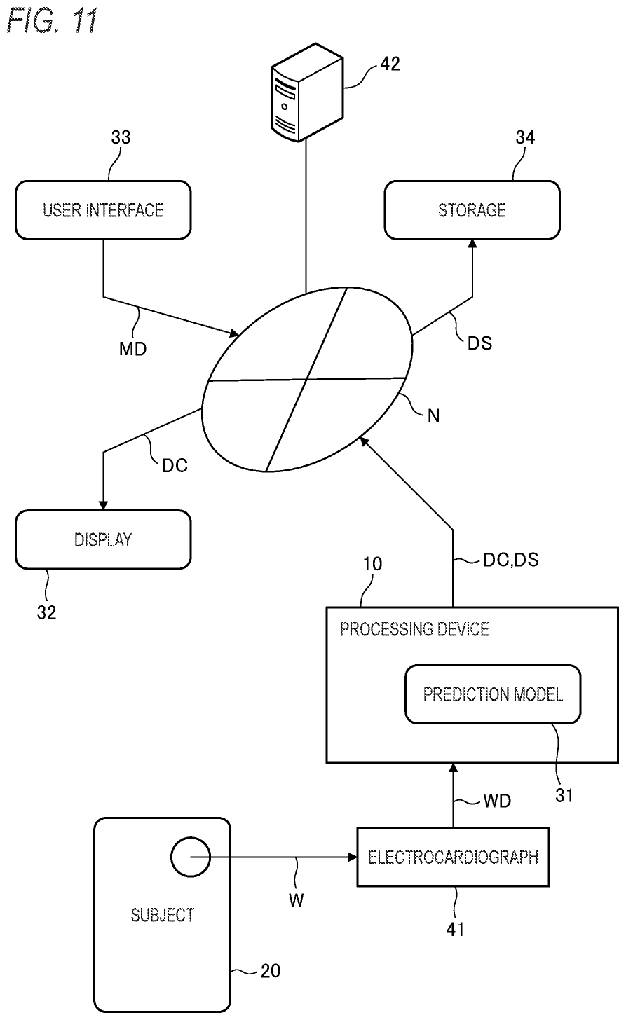

FIG. 11 illustrates other example of the configuration of the processing system 40. The processing device 10 according to the present example is an independent device communicably connected to the electrocardiograph 41. Communication between the electrocardiograph 41 and the processing device 10 may be performed via the communication network N.

In the present example, the prediction model 31 is mounted on the processing device 10. However, same as or similarly to the example described with reference to FIG. 10, the prediction model 31 may be mounted on the server device 42.

Each of the display device 32, the user interface 33 and the storage 34 is communicably connected to the processing device 10 via a communication network N. However, the display device 32 may be mounted on the electrocardiograph 41 or may be mounted on the processing device 10. The user interface 33 may be mounted on the display device 32, may be mounted on the electrocardiograph 41, or may be mounted on the processing device 10. The storage 34 may be mounted on the electrocardiograph 41, may be mounted on the processing device 10, or may be mounted on the server device 42.

In a case where the prediction model 31 is mounted on the server device 42, the prediction model 31 can be shared by a plurality of the processing devices 10. The ease of integrated management of the prediction model 31 in this case becomes more remarkable as the number of processing devices 10 shared increases.

In a case where a function of the model generation device 35 described with reference to FIG. 9 is implemented in the server device 42, the machine learning (including re-learning) of the prediction model 31 can be performed on the server device 42.

As illustrated in FIG. 9, N processing devices 10 (101 to 10N, and N being an integer of 2 or more) may be communicably connected to the storage 34. That is, the data sets DS output from the processing devices 10 may be collectively stored in the storage 34.

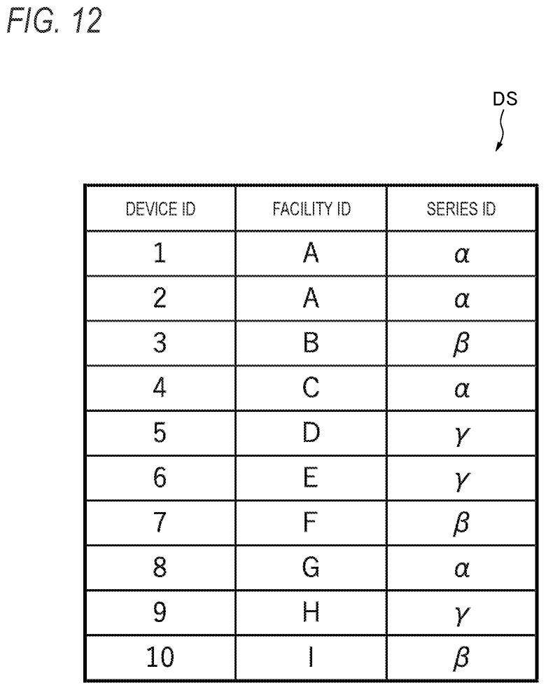

In such a configuration, it is possible to generate and selectively use the prediction model 31 having various features. FIG. 12 illustrates attributes of the plurality of processing devices 10 connectable to the storage 34. In the present example, the data set DS stored in the storage 34 includes a device ID for specifying the processing device 10 that has output the data set DS, a facility ID for specifying a facility in which the processing device 10 is installed, and a series ID for specifying a series group to which the facility belongs.

As an example, the model generation device 35 may perform the machine learning using, as the training data TR, a data set DS output from the processing device 10 having a device ID of 1 and a data set DS output from the processing device 10 having a device ID of 2. These data sets DS are output from the processing device 10 installed in the facility whose facility ID is A.

The data set DS selected in this manner is highly likely to reflect the medical environment in the facility and a determination tendency of the staff member. By performing the machine learning using the data set DS as the training data TR, it is possible to generate the prediction model 31 in which prediction accuracy is enhanced by specializing the facility having the facility ID of A.

In another example, the model generation device 35 may perform the machine learning using, as the training data TR, a data set DS output from the processing device 10 having a device ID of 5, a data set DS output from the processing device 10 having a device ID of 6, and a data set DS output from the processing device 10 having a device ID of 9. All of these data sets DS are output from the processing device 10 installed in the facility whose series ID is γ.

The data set DS selected in this manner is highly likely to reflect the medical environment in the facility belonging to the series and the determination tendency of the staff member. By performing the machine learning using the data set DS as the training data TR, it is possible to generate the prediction model 31 in which the prediction accuracy is enhanced by specializing the facility having the series ID of γ. In addition, a usage method is possible in which the prediction model 31 is used by the processing device 10 that is newly installed in another facility belonging to the same series.

Based on a same or similar idea, the data set DS output from the processing device 10 may include, as attribute information, the scale (the number of sickbeds, the number of staff members, or the like) of the facility in which the processing device 10 is installed. In this case, a usage method is possible in which the prediction model 31 is used by the processing device 10 that is newly installed in a facility having the same scale as the facility serving as the basis of the generated prediction model 31.

Same or similarly, the data set DS output from the processing device 10 may include, as the attribute information, the type (general hospital ward, emergency ward, rehabilitation ward, or the like) of the hospital ward in which the processing device 10 is installed. In this case, a usage method is possible in which the prediction model 31 is used by the processing device 10 that is newly installed in the same type of facility as the facility that is the basis of the generated prediction model 31.

Alternatively, the data set DS output from the processing device 10 may include, as the attribute information, a situation in which the data set is acquired (at the time of health diagnosis, at the time of hospitalization, an examination purpose, an examination target, and the like). In this case, a usage method is possible in which the prediction model 31 is used by the processing device 10 that is newly installed for the purpose of use in the same or similar situation as the situation that is the basis of the generated prediction model 31.

That is, even in a facility or situation in which no clinical data is accumulated, it is possible to provide the advantage of the prediction model 31 generated through the machine learning based on data obtained from a facility or situation having a same or similar attribute tendency.

In another example, the model generation device 35 can perform the machine learning using, as the training data TR, the data sets DS output from all the processing devices 10. In this case, it is possible to generate a prediction model 31 with high versatility.

Each of the processor 12 of the processing device 10 and the processor 352 of the model generation device 35 having various functions described above may be realized by a general-purpose microprocessor that operates in cooperation with a general-purpose memory. Examples of the general-purpose microprocessor can include a CPU, an MPU, and a GPU. Examples of the general-purpose memory can include a ROM and a RAM. In this case, the ROM can store computer programs for realizing the various functions described above. The ROM is an example of a non-transitory computer readable medium configured to store a computer program. The general-purpose microprocessor designates at least part of the programs stored in the ROM, loads the programs in the RAM, and performs the above-described processing in cooperation with the RAM. The computer program may be pre-installed in the general-purpose memory, or may be downloaded from an external server device via the communication network N and then installed in the general-purpose memory. In this case, the external server device is an example of a non-transitory computer readable medium that stores the computer program.

Each of the processor 12 of the processing device 10 and the processor 352 of the model generation device 35 having the various functions described above may be realized by a dedicated integrated circuit such as a microcontroller, an ASIC, or an FPGA capable of executing the computer program described above. In this case, the computer program is pre-installed in a storage element included in the dedicated integrated circuit. The storage element is an example of a computer-readable medium that stores a computer program. Each of the processor 12 of the processing device 10 and the processor 352 of the model generation device 35 having various functions described above may be realized by a combination of a general-purpose microprocessor and a dedicated integrated circuit.

The various configurations described above are merely examples for facilitating the understanding of the presently disclosed subject matter. Each configuration example can be appropriately changed or combined within the scope of the gist of the presently disclosed subject matter.

The index corresponding to the probability that the waveform portion in which the atrial fibrillation is suspected is included in the electrocardiographic waveform W acquired from the subject 20 is not limited to the color of the graphic displayed together with the partial electrocardiographic waveform and the color of the background of the partial electrocardiographic waveform. The color of the partial electrocardiographic waveform itself can also be used as an index. A character or a symbol displayed together with the partial electrocardiographic waveform may be used as an index.

The heart disease used for the prediction by the processing device 10 is not limited to the atrial fibrillation. Examples of the other heart diseases can include premature atrial contraction, paroxysmal supraventricular tachycardia, premature ventricular contraction, ventricular fibrillation, and myocardial infarction. The type of beat information used in the training data TR used for generation of the prediction model 31 is appropriately selected so as to be able to specify a heart disease used for prediction.

The configurations listed below also constitute a part of the presently disclosed subject matter.

(1) A processing device including:
an interface configured to receive electrocardiographic waveform data corresponding to an electrocardiographic waveform of a subject; and
a processor configured to:
   divide the electrocardiographic waveform into a plurality of partial electrocardiographic waveforms, based on the electrocardiographic waveform data;
   calculate a probability that a waveform portion in which a heart disease is suspected is included in each of the plurality of partial electrocardiographic waveforms; and
   cause a display to display an index together with information, the index corresponding to the probability, the information corresponding to the partial electrocardiographic waveform from which the probability is calculated.

(2) The processing device according to the above described (1),
in which the index is a color of the partial electrocardiographic waveform, a color of a background of the partial electrocardiographic waveform, or a color of a graphic displayed together with the partial electrocardiographic waveform.

(3) The processing device according to the above described (1) or (2),
in which the information corresponding to the partial electrocardiographic waveform includes a tachogram based on an R-R interval in the electrocardiographic waveform.

(4) The processing device according to any one of the above described (1) to (3),
in which the interface is configured to receive an instruction to change the index, and
in which the processor is configured to change the index, based on the instruction.

(5) The processing device according to the above described (4),
in which the interface is configured to receive an instruction to designate a specific time section in the electrocardiographic waveform displayed on the display, and
in which the processor is configured to change the index included in the time section, in accordance with an occupancy rate, in the time section, of the partial electrocardiographic waveform displayed together with the index.

(6) The processing device according to the above described (4),
in which the interface is configured to receive an instruction to designate a specific time section in the electrocardiographic waveform displayed on the display, and
in which the processor is configured to:
   specify a first partial electrocardiographic waveform that is similar to a second partial electrocardiographic waveform, the first partial electrocardiographic waveform being the partial electrocardiographic waveform that is not included in the time section, the second partial electrocardiographic waveform being the partial electrocardiographic waveform that is included in the time section, and
   change the index displayed together with the first partial electrocardiographic waveform to the index that is identical to the index displayed together with the second partial electrocardiographic waveform.

(7) The processing device according to any one of the above described (4) to (6),
in which the processor is configured to generate a data set in which data and a part of the electrocardiographic waveform data are associated with each other, the data corresponding to the index changed based on the instruction, the electrocardiographic waveform data corresponding to the partial electrocardiographic waveform specified by the index changed based on the instruction.

(8) The processing device according to any one of the above described (1) to (8),
in which the probability is calculated by inputting data to a prediction model, the data being acquired based on the electrocardiographic waveform, the prediction model being generated through machine learning using a plurality of electrocardiographic waveforms that is determined to include the waveform portion in which the heart disease is suspected.

(9) A non-transitory computer readable storage medium storing a computer program, the computer program including instructions which, when executed by a processor mounted on a processing device, cause the processing device to:
receive electrocardiographic waveform data corresponding to an electrocardiographic waveform of a subject;
divide the electrocardiographic waveform into a plurality of partial electrocardiographic waveforms, based on the electrocardiographic waveform data;
calculate a probability that a waveform portion in which a heart disease is suspected is included in each of the plurality of partial electrocardiographic waveforms; and
cause a display to display an index together with information, the index corresponding to the probability, the information corresponding to the partial electrocardiographic waveform from which the probability is calculated.

(10) A method for generating training data for machine-learning a prediction model, the prediction model being used for calculating a probability that a waveform portion in which a heart disease is suspected is included in an electrocardiographic waveform acquired from a subject, the method including:
acquiring electrocardiographic waveform data corresponding to the electrocardiographic waveform including the waveform portion in which the heart disease is suspected;
acquiring beat information on the subject from the electrocardiographic waveform; and
generating a data set in which the electrocardiographic waveform data is associated with data corresponding to the beat information.

(11) A method for generating a prediction model, the prediction model being used for calculating a probability that a waveform portion in which a heart disease is suspected is included in an electrocardiographic waveform acquired from a subject, the method including:
acquiring training data generated by the method according to the above described (10); and
performing machine learning using the training data.

(12) A method for generating a prediction model, the prediction model being used for calculating a probability that a waveform portion in which a heart disease is suspected is included in an electrocardiographic waveform acquired from a subject, the method including:
acquiring a data set generated by the processing device according to the above described (7); and
performing machine learning using the data set.

(13) The method for generating a prediction model according to the above described (12), further including:
acquiring the data set from a plurality of the processing devices.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A processing device comprising:
an interface configured to receive electrocardiographic waveform data corresponding to an electrocardiographic waveform of a subject; and
a processor configured to:
divide the electrocardiographic waveform into a plurality of partial electrocardiographic waveforms, based on the electrocardiographic waveform data;
calculate a probability that a waveform portion in which a heart disease is suspected is included in each of the plurality of partial electrocardiographic waveforms; and
cause a display to display an index together with information, the index corresponding to the probability, the information corresponding to the partial electrocardiographic waveform from which the probability is calculated.

2. The processing device according to claim 1,
wherein the index is a color of the partial electrocardiographic waveform, a color of a background of the partial electrocardiographic waveform, or a color of a graphic displayed together with the partial electrocardiographic waveform.

3. The processing device according to claim 1,
wherein the information corresponding to the partial electrocardiographic waveform includes a tachogram based on an R-R interval in the electrocardiographic waveform.

4. The processing device according to claim 1,
wherein the interface is configured to receive an instruction to change the index, and
wherein the processor is configured to change the index, based on the instruction.

5. The processing device according to claim 4,
wherein the interface is configured to receive an instruction to designate a specific time section in the electrocardiographic waveform displayed on the display, and
wherein the processor is configured to change the index included in the time section, in accordance with an occupancy rate, in the time section, of the partial electrocardiographic waveform displayed together with the index.

6. The processing device according to claim 4,
wherein the interface is configured to receive an instruction to designate a specific time section in the electrocardiographic waveform displayed on the display, and
wherein the processor is configured to:
specify a first partial electrocardiographic waveform that is similar to a second partial electrocardiographic waveform, the first partial electrocardiographic waveform being the partial electrocardiographic waveform that is not included in the time section, the second partial electrocardiographic waveform being the partial electrocardiographic waveform that is included in the time section, and
change the index displayed together with the first partial electrocardiographic waveform to the index that is identical to the index displayed together with the second partial electrocardiographic waveform.

7. The processing device according to claim 4,
wherein the processor is configured to generate a data set in which data and a part of the electrocardiographic waveform data are associated with each other, the data corresponding to the index changed based on the instruction, the electrocardiographic waveform data corresponding to the partial electrocardiographic waveform specified by the index changed based on the instruction.

8. The processing device according to claim 1,
wherein the probability is calculated by inputting data to a prediction model, the data being acquired based on the electrocardiographic waveform, the prediction model being generated through machine learning using a plurality of electrocardiographic waveforms that is determined to include the waveform portion in which the heart disease is suspected.

9. A non-transitory computer readable storage medium storing a computer program, the computer program comprising instructions which, when executed by a processor mounted on a processing device, cause the processing device to:
receive electrocardiographic waveform data corresponding to an electrocardiographic waveform of a subject;
divide the electrocardiographic waveform into a plurality of partial electrocardiographic waveforms, based on the electrocardiographic waveform data;
calculate a probability that a waveform portion in which a heart disease is suspected is included in each of the plurality of partial electrocardiographic waveforms; and
cause a display to display an index together with information, the index corresponding to the probability, the information corresponding to the partial electrocardiographic waveform from which the probability is calculated.

10. A method for generating training data for machine-learning a prediction model, the prediction model being used for calculating a probability that a waveform portion in which a heart disease is suspected is included in an electrocardiographic waveform acquired from a subject, the method comprising:
acquiring electrocardiographic waveform data corresponding to the electrocardiographic waveform including the waveform portion in which the heart disease is suspected;
acquiring beat information on the subject from the electrocardiographic waveform; and
generating a data set in which the electrocardiographic waveform data is associated with data corresponding to the beat information.

11. A method for generating a prediction model, the prediction model being used for calculating a probability that a waveform portion in which a heart disease is suspected is included in an electrocardiographic waveform acquired from a subject, the method comprising:
acquiring training data generated by the method according to claim 10; and
performing machine learning using the training data.

12. A method for generating a prediction model, the prediction model being used for calculating a probability that a waveform portion in which a heart disease is suspected is included in an electrocardiographic waveform acquired from a subject, the method comprising:
acquiring a data set generated by the processing device according to claim 7; and
performing machine learning using the data set.

13. The method for generating a prediction model according to claim 12, further comprising:
acquiring the data set from a plurality of the processing devices.
